(19) United States
(12) Patent Application Publication
(10) Pub. No.: US 2019/0289886 A1
(43) Pub. Date: Sep. 26, 2019

(54) SUBSTANCE MIXTURES

(71) Applicant: SYMRISE AG, Holzminden (DE)

(72) Inventors: Johannes KIEFL, Holzminden (DE); Susanne PAETZ, Höxter (DE); Jakob LEY, Holzminden (DE); Gerhard KRAMMER, Holzminden (DE); Thomas RIESS, Holzminden (DE); Kathrin LANGER, Dassel-Hilwartshausen (DE); Günter KINDEL, Höxter (DE); Morine VERWOHLT, Linnenkamp (DE); Torsten GEISSLER, Einbeck (DE); Egon GROSS, Holzminden (DE)

(21) Appl. No.: 15/780,346

(22) PCT Filed: Dec. 1, 2015
(86) PCT No.: PCT/EP2015/078255

§ 371 Date: Sep. 27, 2018

(51) Int. Cl. A23L 27/10; A23L 2/56; A23L 27/30; A23L 2/60; A23L 27/00; A61K 9/00; A61K 47/26; A61K 47/10; A61K 47/24

(52) U.S. Cl. CPC A23L 27/10; A23V 2250/628; A23L 27/34; A23L 2/60; A23L 27/84; A23L 27/35; A61K 9/0053; A61K 47/26; A61K 47/10; A61K 47/24; A23V 2002/00; A23V 2200/15; A23V 2200/16; A23V 2250/2132; A23V 2250/61; A23L 2/56

(57) ABSTRACT

Proposed are mixtures of substances containing
(a) phloretin,
(b) naringenin,
(c) at least one further sweet substance other than phloretin and naringenin, and optionally
(d) at least one flavoring substance.

### FIELD OF INVENTION

The invention is in the field of foodstuffs, especially flavoring substances with sweet-optimizing properties and concerns preparations containing phloretin and another flavoring substance, which compensates the undesirable side notes of this substance.

### STATE OF THE ART

The food industry is increasingly developing products that enable consumers to take up valuable components such as flavonoids to support maintenance of health. These products usually contain a reduced sugar content in order to achieve a calorie reduction in the foodstuff. However, the sugar reduction is accompanied by a loss of sweet impression and body as well as mouthfulness of the foodstuff. Therefore, substances such as phloretin are often used to compensate for the missing sweet taste. However, side notes such as unwanted bitterness, astringency and/or lack of mouthfulness thereby arise.

Products containing dihydrochalcones such as phloretin for sweet optimization are known from the state of the art (e.g. EP 2868209 A1 (SYMRISE)). Also known are processes for the production of these substances by extractive, biotechnological and chemical processes (EP 2742983 A1 (SYMRISE); EP 2692729 A1 (SYMRISE), CN 103230408 A1 (FOSHAN)). In addition, there are activities to increase the content of phloretin in extracts by enrichment methods (CN 104292094 (GUILIN)).

Mixtures consisting of phloretin and other flavonoids such as naringenin are for instance used to positively influence uptake of flavonoids in the intestine (WO 0234073). Also known are processes for the extraction of naringenin from citrus fruits such as grapefruit (CN 104277024 (GUILIN)).

Mixtures of flavonoids with phloretin to mask the bitter aftertaste of sweeteners such as steviolgylcosides are also described (EP 2220945 A1). However, the sweet component with a bitter aftertaste is not phloretin but other sweeteners such as thaumatin, neohesperidin dihydrochalcone and the like. Furthermore, in this case phloretin is used to mask other substances and is not masked itself.

Hydroxyflavones are also described to mask the bitter taste (EP 1258200 A1). Sweetened drinks are known from WO 2015 169769 A1 (FIRMNENICH), to which 30 to 200 ppm naringenin is added to increase the sweetening effect. In EP 2923584 A1 (IFF) it is proposed to increase the sweetness of a sweet taste modulator by adding naringenin. The combination of naringenin and phloretin is not explicitly mentioned in these texts. In addition, the naringenin described below offers the additional advantage of rounding the taste profile and a sugar-like taste compared to the use of phloretin.

Despite the sweet-optimizing properties of phloretin, sugar-reduced preparations containing phloretin have a bitter, astringent aftertaste or off-taste.

The effect of a substance for sweet optimization is characterized in that, in combination with at least one other sweet substance, which is present in a concentration which in itself already causes a clearly perceptible sweet intensity, it causes an increased instant sweetness, an increased sweet intensity, an increased mouthfulness and thereby a lower bitterness and lower astringency.

The objective of the present invention was therefore, on the one hand, to round off the sensory profile of phloretin, which is not perfect in taste, and to reduce the bitter aftertaste and, on the other hand, to provide mixtures of substances and flavor preparations that have both a high sweet optimization potential and a rounded taste profile.

### DESCRIPTION OF THE INVENTION

A first object of the invention concerns mixtures of substances containing
(a) phloretin,
(b) naringenin,
(c) at least one further sweet substance different from phloretin and naringenin, and optionally
(d) at least one flavoring substance.

Surprisingly, it was found that the mixtures have an optimized sweetening effect and are particularly suitable for rounding off and optimizing the profile of the sweet taste of foodstuffs. In particular, in combination with naringenin and phloretin, the bitter to pungent aftertaste of sweet substances is masked and at the same time the instant sweetness and sweet intensity are increased. The mixtures can also be expanded into flavoring compositions by adding further flavoring substances. Both, the mixtures of substances and the flavoring composition, are ideally suited to be incorporated into foodstuffs.

Phloretin

Phloretin is mainly obtained from apple plants (Malus sp.) by extraction, whereby biotechnological methods of production are also known (EP 2692729). In plants, phloretin (formula I) is present in the form of the 2-β-glucoside phlorizin and can be extracted as such and, subsequently or in one step, cleaved. Phloretin is a dihydrochalcone belonging to the class of polyketides and closely related to flavonoids.

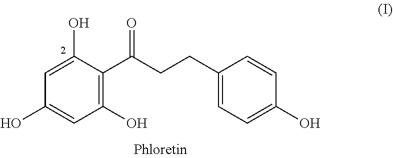 [SMILES: O=C(CCc1ccc(O)cc1)c1c(O)cc(O)cc1O]

Phloretin itself has only a weak sweetening effect, however, can intensify the sweet taste impression of other sweet substances, as described in EP 1998636 B1. However, in addition to the also known bitter-masking property (cf. EP 1972203 B1) against other bitter substances, it shows an inherent bitterness, in particular at higher concentrations, e.g. at 50 ppm. The substance itself can be produced in a manner known per se by extraction of the leaves, branches, root bark or fruits of Malus sp. e.g. using aqueous ethanol and subsequent hydrolysis e.g. using enzymes, acidic ion exchangers, mineral and/or organic acids, as described in EP 2017272 B1, and recovered by subsequent purification by e.g. chromatographic processes.

Naringenin

Naringenin (formula II) is mainly obtained in the form of its glycosides, e.g. naringin, narirutin and prunin, from citrus fruits of the genus Citrus sp., mainly from grapefruit (Citrus paradisi), bitter orange (Citrus x aurantium L.) or bergamot (Citrus bergamia), lemon (Citrus medica, Citrus limon), orange (Citrus sinensis), and as such has no noticeable taste properties. The enzymatic reaction mainly forms S-enantiomer in plants. In the extractive production of naringin, the chiral center of the aglycon can be completely or at least partially racemized, so that partially or completely racemic naringenin is obtained in a subsequent glycoside cleavage.

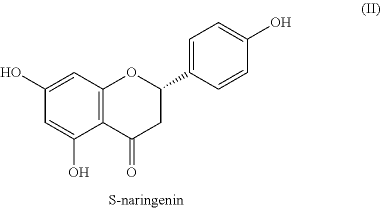 [SMILES: O=C1C[C@@H](c2ccc(O)cc2)Oc2cc(O)cc(O)c21]

The content of naringenin respectively naringin differs in different citrus species and varies according to growing area, plant variety, plant part and state of ripeness. Naringenin, for example, is released during juice production or storage, so that a small proportion is found in ready-to-eat products. Naringenin in free form is not present in orange, lemon or grapefruit juices in a concentration sufficient for the effect according to the invention (>5 ppm). Thus, in Yanez, J. A.; Remsberg, C. M.; Miranda, N. D.; Vega-Villa, K. R.; Andrews, P. K.; Davies, N. M., Pharmacokinetics of selected chiral flavonoids: hesperetin, naringenin and eriodictyol in rats and their content in fruit juices. Biopharmaceutics & Drug Disposition 2008, 29, (2), 63-82, naringenin was found in orange juices with a maximum of 0.5 ppm, in grapefruit juices up to approx. 2.06 ppm, which, however, is a concentration that is regularly too low to develop a sweet-optimizing effect, in particular because a 250-fold concentration of very bitter Naringin is found in the same juices.

Naringenin, which is preferred for the purposes of the present invention, can be both R-naringenin and S-naringenin as well as mixtures of the two enantiomers. Naringenin, for the purposes of the present invention, is preferably present as a mixture of R- and S-naringenin, whereby about 0.01 to about 99.9, preferably about 50 to 99.5, more preferably 90 to 99% by weight, of S-naringenin based on the total amount of naringenin can be present.

These mixtures can be obtained from one or more of the following flavonoids by the enzymatic or fermentative process of EP 2692729 A1 described further above: Naringenin, naringin, narirutin. As far as these texts describe the production of the mixtures, their content is hereby included by reference.

Sweet Substances

Sweet-tasting carbohydrates and especially sugar (sucrose), such as sucrose, trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-glycerolaldehyde, or maltodextrins of various degrees of polymerization can be used as sweet substances or sweet-tasting substances in the first place. Also suitable are plant preparations containing these substances, for example based on sugar beet (Beta vulgaris ssp., sugar fractions, sugar syrup, molasses), sugar cane (Saccharum officinarum ssp., molasses, sugar cane syrup), honey, maple syrup (Acer ssp.) or agaves (agave syrup).

Also possible are
(i) synthetic, i.e. usually enzymatically produced starch or sugar hydrolysates (invert sugar, fructose syrup);
(ii) fruit concentrates (e.g. based on apples or pears);
(iii) sugar alcohols (e.g. erythritol, threitol, arabitol, ribotol, xylitol, sorbitol, mannitol, dulcitol, lactitol);
(iv) sweet-tasting proteins and peptides (e.g. miraculin, monellin, thaumatin, curculin, brazzein);
(v) sweeteners (e.g. Magap, sodium cyclamate, acesulfame K, neohesperidine dihydrochalcone, saccharin sodium salt, aspartame, superaspartame, neotame, alitame, sucralose, steviosides, rebaudiosides, rubusosides, suaviosides, mogrosides, lugdunames, carrelames, sucrononates, sucrooctates, monon, phenylodulcin, phyllodulcin, abrusosides (e.g. described in WO 2015/091298));
(vi) naturally occurring sweeteners selected from the subgroup consisting of stevioside, steviol bioside, rebaudioside A, further steviol glycosides such as rebaudioside B, rebaudioside C, rebaudioside D, rebaudioside E, rebaudioside F, rebaudioside G, rebaudiosid H, dulcoside and/or rubusoside, oslandin, polypodoside A, strogin 1, strogin 2, strogin 4, selligueanin A, dihydroquercetin-3-acetate, perillartin, telosmoside A15, periandrin I-V, phyllodulcin, pterocaryosides, cyclocaryosides, nnucuroziosides, transanethole, trans-cinnamaldehyde, bryosides, bryonosides, bryonodulcosides, carnosiflosides, scandosides, gypenosides, trilobatin, phloridzin, dihydroflavanols, hematoxylin, cyanin, chlorogenic acid, albiziasaponin, telosmosides, gaudichaudioside, mogrosides, hernandulcin, glycyrrhetinic acid, balansin A, balansin B;
(vii) the naturally occurring sweeteners from group (i) in the form of extracts or enriched fractions of these extracts, in particular Thaumatococcus extracts (cat treat), extracts from Stevia ssp. (in particular Stevia rebaudiana), swingle extract (Momordica or Siratia grosvenorii, Luo-Han-Guo), extracts from liquorice root, also Glycerrhyzia ssp. (esp. Glycerrhyzia glabra), Rubus ssp. (especially Rubus suavissimus), citrus extracts, extracts from Hydrangea macrophylla var. thunbergii Makino, extracts from Lippia dulcis, extracts from Mycetia balansae and correspondingly enriched fractions of these extracts may also be used in the flavoring mixtures according to the present invention;
(viii) sweet-tasting amino acids (e.g. glycine, D-leucine, D-threonine, D-asparagine, D-phenylalanine, D-tryptophan, L-proline);
(viii) further sweet-tasting low molecular weight substances, such as hernandulcin, dihydrochalconglycosides, glycyrrhizin, glycerrhetinic acid, their derivatives and salts, extracts of liquorice (Glycyrrhizza glabra ssp.), Lippia dulcis extracts, Momordica ssp. extracts or
(ix) fruit preparations or extracts e.g. from Momordica grosvenori [Luo Han Guo], Hydrangea dulcis as described in EP 2298084 B1 or Stevia ssp. (e.g. Stevia rebaudiana) extracts
(x) enzymatically treated glycosidic sweeteners or extracts from Stevia ssp. or Rubus ssp. as described in WO 2015/062998.
(xi) extracts or isolated balansins from Mycetia balansae as described in WO 2012/164062.

For technical reasons, it is preferred that these sweet substances or mixtures thereof in oral preparations achieve a sweet intensity corresponding to at least a sweet intensity of a comparative oral preparation containing only 2% sucrose without other sweet substances.

Flavoring Substances

In a preferred embodiment of the present invention, flavoring substances are contained as optional component (d).

Suitable flavoring substances may be selected from the list below: Acetophenone, allyl capronate, alpha-ionone, beta-ionone, anisaldehyde, anisyl acetate, anisyl formate, benzaldehyde, benzothiazole, benzyl acetate, benzyl alcohol, benzyl benzoate, butyl butyrate, butyl capronate, butylidenephthalide, carvone, camphene, caryophyllene, cineol, cinnannyl acetate, citral, citronellol, citronellal, citronellyl acetate, cyclohexyl acetate, cymene, damascon, delta-decalactone, diacetyl, dihydrocoumarin, dimethylanthranilate, dodecalactone, acetic acid, ethyl acetate, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerianate, ethyl lactate, ethyl methyl butyrate, ethyl propionate, eucalyptol, eugenol, ethyl heptylate, geraniol, gerany acetate, methyl dihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-3-hexenylacetate, cis-3-hexenyl capronate, trans-2-hexenyl capronate, cis-3-hexenyl formate, para-hydroxybenzyl acetone, isoamyl alcohol, isoamyl isovalerianate, isobutyl butyrate, isobutyraldehyde, isoeugenol methyl ether, isopropyl methyl thiazole, lauric acid, leavulinic acid, linalool, linalool oxide, linalyl acetate, menthol, menthofuran, methylanthranilate, methyl-butanol, methylbutyric acid, 2-methyl butyl acetate, methyl capronate, methyl cinnamate, 5-methylfurfural, 3,2,2-methylcyclopentenolone, 6,5,2-methylheptenone, methyl jasmonate, 2-methylmethylbutyrate, 2-methyl-2-pentenolic acid, methyl thiobutyrate, 3,1-methylthiohexanol, 3-nnethylthiohexyl acetate, nerol, nerylacetate, trans,trans-2,4-nonadienal, 2,4-nonadienol, 2,6-nonadienol, nootkatone, delta octalactone, gamma octalactone, 2-octanol, 3-octanol, 1,3-octenol, 1-octylacetate, 3-octylacetate, palmitic acid, paraldehyde, phellandrene, pentanedione, phenylethyl acetate, phenylethyl alcohol, phenylethyl isovalerianate, propionaldehyde, propylbutyrate, pulegone, pulegol, sinensal, sulfurol, terpinene, terpineol, terpinols, 8,3-thiomenthanone, 4,4,2-thiomethylpentanone, thymol, delta-undecalactone, gamma-undecalactone, valencene, valeric acid, vanillin, acetoin, ethyl vanillin, ethyl vanillin isobutyrate (=3-ethoxy-4-isobutyryloxybenzaldehyde), 2,5-dimethyl-4-hydroxy-3(2H)-furanone and its derivatives (preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (preferably ethyl maltol), coumarin and coumarin derivatives, gamma-lactones (preferably gamma-undecalactone, gamma-nonalactone, gamma-decalactone), delta-lactones (preferably 4-methyldelta-decalactone, massoilactone, delta-decalactone, tuberolactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)furanone, acetic acid isoamyl ester, butyric acid isoamyl ester, 3-methyl butyric acid ethyl ester, ethyl-3-methyl-3-phenylglycidate, ethyl-2-trans-4-cis-decadienoate, 1,1-dimethoxy-2,2,5-trimethyl-4-hexane, phenylacetaldehyde, 2-methyl-3-(methylthio)furan, 2-methyl-3-furanthiol, bis(2-methyl-3-furyl)disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethyl pyrazine, 2-ethyl-3,5-dimethyl pyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetyl pyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, guaiacol, 3-hydroxy-4,5-dimethyl-2(5H)-furanone, 3-hydroxy-4-methyl-5-ethyl-2(5H)-furanone, cinnamic aldehyde, cinnamic alcohol, methyl salicylate, isopulegol and (not explicitly mentioned here) stereoisomers, enantiomers, positional isomers, diastereomers, cis/trans-isomers or epimers of these substances.

Typical examples are further: alpha-ionone, beta-ionone, benzaldehyde, citral, damascon, delta-decalactone, diacetyl, dimethylanthranilate, dodecalactone, acetic acid, ethyl acetate, ethyl butyrate, ethyl furaneol, ethyl isobutyrate, ethyl isovalerianate, ethyl methyl butyrate, ethyl propionate, geraniol, geranyl acetate, heliotropin, trans-2-hexenal, cis-3-hexenol, cis-3-hexenyl acetate, para-hydroxybenzyl acetone, isobutyraldehyde, linalool, linalyl acetate, 3,2,2-methylcyclopentenolone, 2-nnethylnnethyl butyrate, nerol, neryl acetate, vanillin, ethyl vanillin, 2,5-dimethyl-4-hydroxy-3(2H)-furanone, homofuronol (=2-ethyl-5-methyl-4-hydroxy-3(2H)-furanone and 5-ethyl-2-methyl-4-hydroxy-3(2H)-furanone), maltol and maltol derivatives (preferably ethyl maltol), gamma-lactones (preferably gamma-undecalactone, gamma-nonalactone, gamma-decalactone), delta-lactones (preferably 4-methyldelta-decalactone, massoilactone, delta-decalactone, tuberolactone), 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, acetic acid isoamyl ester, butyric acid isoamyl ester, 3-methyl-butyric acid ethyl ester, ethyl-2-trans-4-cis-decadienoate, (E,E)-2,4-decadienal, guaiacol, cinnamic aldehyde and stereoisomers, enantiomers, position isomers, diastereomers, cis/transisomers or epimers of these substances.

Component (d) may also be modulating aroma and/or flavoring substances, preferably selected from the group consisting of adenosine 5′-monophosphate, cytidine-5′-monophosphate, inosine 5′-monophosphate, and pharmaceutically acceptable salts thereof; lactisols; 2,4-dihydroxybenzoic acid; 3-hydroxybenzoic acid; sodium salts, preferably sodium chloride, sodium lactate, sodium citrate, sodium acetate, sodium gluconoate; hydroxyflavanones, such as eriodictyol, homoeriodictyol, and sodium salts thereof; hydroxybenzoic acid amides, such as 2,4-dihydroxybenzoic acid vanillylamide, 2,4-dihydroxybenzoic acid N-(4-hydroxy-3-methoxybenzyl)amide, 2,4,6-trihydroxybenzoic acid-N-(4-hydroxy-3-methoxybenzyl)amide, 2-hydroxybenzoic acid-N-4-(hydroxy-3-methoxybenzyl)amide, 4-hydroxybenzoic acid-N-(4-hydroxy-3-methoxybenzyl)-amide, 2,4-dihydroxybenzoic acid-N-(4-hydroxy-3-methoxy-benzyl)-amide monosodium salt, 2,4-dihydroxybenzoic acid-N-2-(4-hydroxy-3-methoxy-phenyl)-ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N42-(4-hydroxy-3-methoxyphenyl)ethyllamide; 4-hydroxy-benzoic acid vanillylamide; hydroxydeoxybenzoins, such as 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl) ethanone; hydroxyphenlaalkanediones, such as gingerdione-[2], gingerdione-[3], gingerdione-[4], dehydrogingerdione-[2], dehydrogingerdione-[3], dehydrogingerdione-[4]); diacetyl trimers; y-amino butyric acids and divanillins; bicyclo[4.1.0]heptane-7-carboxamides; cyclopropanecarboxylic acid(3-methyl-cyclohexyl)amides, aromatic neo-menthylamides; geranylamine derivatives of oxalic acid and neomenthyl derivatives.

Mixture of Substances

The mixtures of substances according to the invention may comprise components (a) and (b) in a weight ratio of about 99:1 to about 1:99. It is particularly preferred if component (b) is a mixture of S- and R-naringenin, for example in a weight ratio of 99:1 to 80:20 and in particular about 97:3 to about 90:10.

Also preferred are mixtures of substances containing
(a) about 1 to about 25% by weight phloretin
(b) about 1 to about 25% by weight of naringenin
(c) about 1 to about 50% by weight of at least one further sweet substance other than phloretin and naringenin,
   with the proviso that the amounts given sum up to 100% by weight, optionally by the addition of flavoring substances that constitute optional component (d).

A typical composition of the mixtures in terms of components (a) and (b) is therefore as follows:
(a) about 5 to about 99.8, preferably about 25 to 75, more preferably 40 to 60% by weight phloretin,
(b1) about 0.1 to about 94.9, preferably about 25 to 75, more preferably 40 to 60% by weight of S-naringenin, wherein one of the naringenin enantiomers may be present by 100:1 to 1:100 based on the total naringenin content,
(b2) about 0.1 to about 94.9, preferably about 25 to 75, more preferably 40 to 60% by weight of R-naringenin, wherein one of the naringenin enantiomers may be present by 100:1 to 1:100 based on the total naringenin content,
   with the proviso that the amounts given sum up to 100% by weight.

Particularly advantageous are mixtures that correspond to the previous composition and wherein
(a) about 5 to about 90, preferably between 25 to 75, more preferably 45 to 55 wt. % phloretin,
(b1) between 10 to about 85, preferably between 30 to 70, more preferably 45 to 55 wt. % S-naringenin,
(b2) between 0.1 to about 85, preferably between 0.1 to 50, more preferably 0.1 to 1% by weight of R-naringenin,
   are included.

Even more advantageous are mixtures that correspond to the previous composition and wherein
(a) more than 30% by weight phloretin,
(b1) more than 30% by weight S-naringenin,
(b2) less than 1% by weight of R-naringenin are included.

Preferred embodiments also include mixtures of substances containing components (a+b) and (c) in a weight ratio of about 20:80 to 80:20, preferably about 40:60 to 60:40 and in particular about 50:50.

These mixtures can be obtained from one or more of the following flavonoids by the enzymatic or fermentative method of EP 2692729 described further above: Naringenin, Naringin, narirutin. As far as these texts describe the production of the mixtures, their content is hereby included by reference.

Oral Preparations

A further object of the invention relates to oral preparations, in particular foodstuffs containing the mixtures of substances described above with or without content of flavoring substances and preferably with flavoring substances in amounts of 0.00001 to about 2% by weight. Other preferred amounts to be added are 0.0001 to about 1.5% by weight, preferably 0.001 to about 1% by weight, more preferably about 0.01 to about 0.5% by weight and even more preferably about 0.05 to 0.1% by weight.

In particular, the oral preparations in general and the foodstuffs specifically contain about 10 to about 100 ppm phloretin and about 10 to about 100 ppm naringenin.

Typical examples of oral preparations include:
bakery products such as bread, dry biscuits, cakes, other pastries,
confectionery (e.g. chocolate, chocolate bar products, other bar products, fruit gum, hard and soft caramels, chewing gum),
alcoholic or non-alcoholic beverages (e.g. coffee, tea, iced tea, wine, wine-based beverages, beer, beer-containing beverages, liqueurs, spirits, brandies, (carbonated) fruit-containing lemonades, (carbonated) isotonic beverages, (carbonated) soft drinks, nectars, spritzers, fruit and vegetable juices,
fruit or vegetable juice preparations,
instant beverages (e.g. instant cocoa beverages, instant tea beverages, instant coffee beverages, instant fruit beverages),
meat products (e.g. ham, fresh sausage or raw sausage preparations, seasoned or marinated fresh or salted meat products),
eggs or egg products (dried egg, egg white, egg yolk), cereal products (e.g. breakfast cereals, muesli bars, pre-cooked rice products),
dairy products (e.g. dairy drinks, buttermilk drinks, milk ice cream, yoghurt, kefir, cream cheese, soft cheese, hard cheese, dried milk powder, whey, whey drinks, butter, buttermilk, partially or completely hydrolyzed products containing milk protein),
products made from soy protein or other soybean fractions (for example soy milk and products made therefrom, fruit beverages containing soy protein, preparations containing soy lecithin, fermented products such as tofu or tempeh or products made therefrom),
products from other vegetable protein sources, such as oat protein drinks,
fruit preparations (e.g. jams, fruit ice cream, fruit sauces, fruit fillings),
vegetable preparations (e.g. ketchup, sauces, dried vegetables, frozen vegetables, pre-cooked vegetables, boiled vegetables),
snacks (e.g. baked or fried potato chips or potato dough products, extrudates based on corn or peanuts),
fat and oil-based products or emulsions thereof (e.g. mayonnaise, remoulade, dressings),
other ready-meals and soups (e.g. dry soups, instant soups, pre-cooked soups),
spices, spice mixtures and especially seasonings, which are used, for example, in the snack sector.

confectionery, dairy products and especially non-alcoholic beverages are particularly preferred, with sweetened beverages being preferred.

The oral preparations may also be tooth and oral care products such as toothpastes, mouthwashes or chewing gum.

Food Additives

A further embodiment of the present invention envisages that the oral preparations and especially the foodstuffs may contain further auxiliaries and additives, such as starter cultures, emulsifiers, thickeners, food acids, acidity regulators, vitamins, antioxidants, food colorants and the like in amounts of for example about 0.1 to about 10% by weight, preferably about 0.5 to about 8% by weight, more preferably about 1 to about 5% by weight and even more preferably about 2 to about 3% by weight.

Emulsifiers

Emulsifiers are characterized by their important property of being soluble in both water and fat. Emulsifiers usually consist of a fat-soluble and a water-soluble part. They are used whenever water and oil are to be mixed consistently and homogeneously.

Suitable emulsifiers used in the food processing industry are selected from: Ascorbyl palmitate (E 304), lecithin (E 322), phosphoric acid (E 338), sodium phosphate (E 339), potassium phosphate (E 340), calcium phosphate (E 341), magnesium orthophosphate (E 343), propylene glycol alginate (E 405), polyoxyethylene(8)stearate (E 430), polyoxyethylene stearate (E 431), ammonium phosphatides (E 442), sodium phosphate and potassium phosphate (E 450), sodium salts of fatty acids (E 470 a), mono and diglycerides of fatty acids (E 471), acetic monoglycerides (E 472 a), lactic monoglycerides (E 472 b), citric monoglycerides (E 472 c), tartaric monoglycerides (E 472 d), di-acetyl tartaric monoglycerides (E 472 e), sugar esters of fatty acids (E 473), sugar glycerides (E 474), polyglycerides of fatty acids (E 475), polyglycerol polyricinoleate (E 476), propylene glycol esters of fatty acids (E 477), sodium stearyl lactylate (E 481), calcium stearoyl 2-lactylate (E 482), stearyl tartrate (E 483), sorbitan monostearate (E 491), stearic acid (E 570).

Thickeners

Thickeners are substances that are primarily able to bind water. Withdrawal of unbound water leads to an increase in viscosity. At a concentration characteristic of any thickener, this effect is also accompanied by network effects, which usually lead to a disproportionate increase in viscosity. In this case it is said that molecules ‘communicate’ with each other, i.e. tangle up. Most thickeners are linear or branched macromolecules (e.g. polysaccharides or proteins) that can interact with each other through intermolecular interactions such as hydrogen bonds, hydrophobic interactions or ion relationships. Extreme cases of thickeners are layered silicates (bentonites, hectorites) or hydrated SiO₂ particles, which are dispersed as particles and can bind water in their solid-state structure respectively can interact with each other due to the described interactions. Examples are:
E 400—Alginic acid
E 401—Sodium alginate
E 402—Potassium alginate
E 403—Ammonium alginate
E 404—Calcium alginate
E 405—Propylene glycol alginate
E 406—Agar Agar
E 407—Carrgeen, Furcelleran
E 407—locust bean gum
E 412—Guar gum
E 413—Traganth
E 414—Gunn arabic
E 415—Xanthan
E 416—Karaya (Indian tragacanth)
E 417—Tara gum (Peruvian locust bean gum)
E 418—Gellan
E 440—Pectin, opekta
E 440ii —Amidated pectin
E 460—Microcrystalline cellulose, cellulose powder
E 461—Methyl cellulose
E 462—Ethyl cellulose
E 463—Hydroxypropyl cellulose
E 465—Methyl ethyl cellulose
E 466—Carboxymethylcellulose, sodium carboxymethylcellulose

Food Acids

The foodstuffs may contain carboxylic acids. Acids for the purposes of the invention are preferably acids permitted in foodstuffs, in particular those mentioned here:
E 260—Acetic acid
E 270—Lactic acid
E 290—Carbon dioxide
E 296—Malic acid
E 297—Fumaric acid
E 330—Citric acid
E 331—Sodium citrate
E 332—Potassium citrate
E 333—Calcium citrate
E 334—Tartaric acid
E 335—Sodium tartrate
E 336—Potassium tartrate
E 337—Sodium potassium tartrate
E 338—Phosphoric acid
E 353—Metatartaric acid
E 354—Calcium tartrate
E 355—Adipic acid
E 363—Succinic acid
E 380—Triammonium citrate
E 513—Sulphuric acid
E 574—Gluconic acid
E 575—Glucono-delta-Lactone

Acidity Regulators

Acidity regulators are food additives that keep the acidity or basicity and thus the desired pH value of a food constant. These are mostly organic acids and their salts, carbonates, more rarely also inorganic acids and their salts. The addition of an acidity regulator partly increases the stability and firmness of the food, causes a desired precipitation and improves the effectiveness of preservatives. Unlike acidifiers, they are not used to change the taste of food. Their effect is based on the formation of a buffer system in the food, in which the pH value does not change or only changes slightly when acid or basic substances are added. Examples are:
E 170—Calcium carbonate
E 260-263—Acetic acid and acetates
E 270—Lactic acid
E 296—Malic acid
E 297—Fumaric acid
E 325-327—Lactates (lactic acid)
E 330-333—Citric acid and citrates
E 334-337—Tartaric acid and tartrates
E 339-341—Orthophosphates
E 350-352—Malates (malic acid)
E 450-452—Di-, tri- and polyphosphates
E 500-504—Carbonates (carbonic acid)
E 507—Hydrochloric acid and chlorides
E 513-517—Sulfuric acid and sulfates
E 524-528—Hydroxides E 529-530—Oxides E 355-357—Adipic acid and adipates E 574-578—Gluconic acid and gluconates

Vitamins

In a further embodiment of the present invention, the food additives may contain vitamins as a further optional group of additives. Vitamins have a wide variety of biochemical effects. Some are similar to hormones and regulate the mineral metabolism (e.g. vitamin D), or have an effect on cell and tissue growth and cell differentiation (e.g. some forms of vitamin A). Others are antioxidants (e.g. vitamin E and, under certain circumstances, vitamin C). The largest number of vitamins (e.g. B vitamins) are precursors for enzymatic co-factors that support enzymes in catalyzing certain processes in the metabolism. In this context, vitamins can sometimes be closely bound to the enzymes, for example as part of a prosthetic group: one example is biotin, which is part of an enzyme responsible for the formation of fatty acids. On the other hand, vitamins can also be less strongly bound and then act as co-catalysts, for example as groups that can easily be split off and transport chemical groups or electrons between the molecules. Folic acid, for example, transports methyl, formyl and methylene groups into the cell. Although their support in enzyme-substrate reactions is well known, their other properties are also of great importance for the body.

For the purposes of the present inventions vitamins include substances selected from the group consisting of

Vitamin A (retinol, retinal, beta-carotene),

Vitamin B₁ (thiamine),

Vitamin B₂ (rioflavin),

Vitamin B₃ (niacin, niacinamide),

Vitamin B₅ (panthothenic acid),

Vitamin B₆ (pyridoxine, pyridoxamine, paridoxal),

Vitamin B₇ (biotin),

Vitamin B₉ (folic acid, folic acid),

Vitamin B₁₂ (cyanobalamin, hydroxycobalmin, methylcobalmin),

Vitamin C (ascorbic acid),

Vitamin D (cholecalciferol),

vitamin E (tocopherols, tocotrienols) and

Vitamin K (phyllolquinone, menaquinone).

Besides ascorbic acid, the preferred vitamins are the group of tocopherols.

Antioxidants

Both natural and artificial antioxidants are used in the food industry. Natural and artificial antioxidants differ primarily in the fact that the former occurs naturally in food and the latter is produced artificially. Natural antioxidants are obtained, for example, from vegetable oils if they are to be used as food additives. For example, vitamin E—also known as tocopherol—is often produced from soy oil. Synthetic antioxidants such as propyl gallate, octyl gallate and dodecyl gallate are obtained by chemical synthesis. The gallates can cause allergies in sensitive persons. Further antioxidants that can be used in compositions of the present invention are: sulphur dioxide, E 220 sulphites sodium sulphite, E 221 sodium hydrogen sulphite, E 222 sodium disulphite, E 223 potassium disulphite, E 224 calcium sulphite, E 226 calcium hydrogen sulphite, E 227 potassium hydrogen sulphite, E 228 lactic acid, E 270 ascorbic acid, E 300 sodium L-ascorbate, E 301 calcium L-ascorbate, E 302 ascorbic acid ester, E 304 tocopherol, E 306 alpha-tocopherol, E 307 gamma-tocopherol, E 308 deltatocopherol, E 309 propylgallate, E 310 octygallate, E 311 dodecylgallate, E 312 isoascorbic acid, E 315 sodium isoascorbate, E 316 tertiary butyl hydroquinone (TBHQ), E 319 butylhydroxianisol, E 320 butylhydroxitoluene, E 321 lecithin, E 322 citric acid, E 330 salts of citric acid (E 331 & E 332) sodium citrate, E 331 potassium citrate, E 332 calcium disodium EDTA, E 385 diphosphates, E 450 disodium diphosphate, E 450a trisodium diphosphate, E 450b tetrasodium diphosphate, E 450c dipotassium diphosphate, E 450d tripotassium diphosphate, E 450e dicalcium diphosphate, E 450f calcium dihydrogen diphosphate, E 450g triphosphate, E 451 pentasodium triphosphate, E 451a pentapotassium triphosphate, E 451b polyphosphate, E 452 sodium polyphosphate, E 452a potassium polyphosphate, E 452b sodium calcium polyphosphate, E 452c calcium polyphosphate, E 452d tin-II chloride, E 512.

Food colorants

Food colorants, or colorants in short, are food additives for coloring food. Colorants are divided into the groups of natural colorants and synthetic colorants. The nature-identical colorants are also of synthetic origin. The nature-identical colorants are synthetic replicas of naturally occurring coloring substances. Suitable colorants for use in the present composition are selected from: Curcumin, E 100 riboflavin, lactoflavin, vitamin B2, E 101 tartrazine, E 102 quinoline yellow, E 104 yellow orange S, yellow orange RGL, E 110 cochineal acid, carmine, E 120 azorubin, carmoisin, E 122 amaranth, E 123 cochineal red A, Ponceau 4 R, Victoriascarlet 4 R, E 124 erythrosine, E 127 allura red AC, E 129 patentblue V, E 131 indigotine, indigo carmine, E 132 brilliant blue FCF, patent blue AE, amido blue AE, E 133 chlorophylls, chlorophylline, E 140 copper complexes of chlorophylls, copper chlorophylline complex, E 141 brillant acid green, green S, E 142 sugar liquor, sugar couleur, E 150 a sulphite liquor-sugar, E 150 b ammonia sugar, E 150 c ammonium sulphite sugar cullet, E 150 d brilliant black FCF, brilliant black PN, black PN, E 151 vegetable coal, E 153 brown FK, E 154 brown HT, E 155 carotene, carotene, E 160 a annatto, bixin, norbixin, E 160 b capsanthin, capsorubin, E 160 c lycopin, E 160 d beta-apo-8′-carotene, apocarotene, beta-apocarotene, E 160 e beta-apo-8′-carotenic acid ethyl ester (C30), apocarotene ester, beta-carotenic acid ester, E 160 flutein, xanthophyll, E 161 b canthaxanthin, E 161 g betanin, praying red, E 162 anthocyanins, E 163 calcium carbonate, E 170 titanium dioxide, E 171 iron oxides, iron hydroxides, E 172 aluminium, E 173 silver, E 174 gold, E 175 litholrubin BK, ruby pigment BK, E 180.

Oral and Dental Care Products

Orally consumable, sweet-tasting products according to invention can also be used for oral and dental cleaning and care. Examples are toothpastes, tooth gels, tooth powder, mouthwashes and the like. Toothpastes or toothpastes are generally understood as gel-like or pasty preparations of water, thickeners, humectants, abrasives or cleaning agents, surfactants, sweeteners, flavoring agents, deodorizing agents and active agents against oral and dental diseases. All common cleaning agents, such as chalk, dicalcium phosphate, insoluble sodium metaphosphate, aluminum silicates, calcium pyrophosphate, finely divided synthetic resins, silicas, aluminum oxide and aluminum oxide trihydrate can be used in the toothpastes according to the invention.

Preferably suitable cleaning agents for the toothpastes according to the invention are in particular finely divided xerogelic silicas, hydrogel silicas, precipitated silicas, aluminum oxide trihydrate and finely divided alpha-alumina or mixtures of these cleaning agents in amounts of 15 to 40% by weight of the toothpaste. Mainly low molecular weight polyethylene glycols, glycerol, sorbitol or mixtures of these products in amounts of up to 50% by weight are possible humectants. Among the known thickeners the thickening, finely divided gel silicas and hydrocolloids, e.g. carboxymethylcellulose, hydroxyethylcellulose, hydroxypropylguar, hydroxyethyl starch, polyvinylpyrrolidone, high-molecular polyethylene glycol, vegetable gums such as traganth, agar agar, carragheenmoos, gum arabic, xantham gum and carboxyvinyl polymers (e.g. Carbopol° grades) are suitable. In addition to the mixtures of menthofuran and menthol compounds, the oral and dental care products can contain surface-active substances in particular, preferably anionic and non-ionic high-foaming surfactants, such as those already mentioned above, but in particular alkyl ether sulphate salts, alkyl polyglucosides and mixtures thereof.

Other Common Toothpaste Additives are:
Preservatives and antimicrobial substances such as p-hydroxybenzoic acid methyl, ethyl or propyl esters, sodium sorbate, sodium benzoate, bromochlorophene, phenylsalicylic acid esters, thymol and the like;
anti-calculus agents, e.g. organophosphates such as 1-hydroxyethane-1,1-diphosphonic acid, 1-phosphonopropane-1,2,3-tricarboxylic acid and others known for example from US 3,488,419, DE 2224430 Al and DE 2343196 A1;
other caries-inhibiting substances such as sodium fluoride, sodium monofluorophosphate, tin fluoride;
sweeteners, such as saccharin sodium, sodium cyclamate, sucrose, lactose, maltose, fructose or Aspartam®, (L-aspartyl-L-phenylalanine methyl ester), stevia extracts or their sweetening components, in particular ribeaudiosides;
additional flavors such as eucalyptus oil, anise oil, fennel oil, caraway oil, methyl acetate, cinnamaldehyde, anethole, vanillin, thymol and mixtures of these and other natural and synthetic flavors;
pigments such as titanium dioxide;
colorants;
buffer substances such as primary, secondary or tertiary alkali phosphates or citric acid/sodium citrate;

wound healing and anti-inflammatory substances such as allantoin, urea, azulene, chamomile active ingredients and acetylsalicylic acid derivatives.

A preferred embodiments of the cosmetic preparations are toothpastes in the form of an aqueous, pasty dispersion containing polishing agents, humectants, viscosity-regulators and optionally other usual components, as well as the mixture of menthofuran and menthol compounds in amounts of 0.5 to 2% by weight.

In mouthwashes, a combination with aqueous-alcoholic solutions of various degrees of essential oils, emulsifiers, astringent and toning drug extracts, calculus-inhibiting, antibacterial additives and taste correcting agents is easily possible. Another preferred embodiment of the invention is a mouthwash in the form of an aqueous or aqueous-alcoholic solution containing the mixture of menthofuran and menthol compounds in quantities of 0.5 to 2% by weight. In mouthwashes which are diluted before application, sufficient effects can be achieved with higher concentrations according to the intended dilution ratio.

Hydrotropes, such as ethanol, isopropyl alcohol, or polyols can also be used to improve the flow behavior; these substances largely correspond to the carriers described above. Polyols considered here have preferably 2 to 15 carbon atoms and at least two hydroxyl groups. The polyols can also contain other functional groups, in particular amino groups, respectively be modified with nitrogen. Typical examples are
glycerol;
alkylene glycols, such as ethylene glycol, diethylene glycol, propylene glycol, butylene glycol, hexylene glycol and polyethylene glycols with an average molecular weight of 100 to 1,000 Daltons;
technical oligoglycerol mixtures with a degree of self-condensation of 1.5 to 10, such as technical diglycerol mixtures with a diglycerol content of 40 to 50% by weight;
methyol compounds, in particular trimethylolethane, trimethylolpropane, trimethylolbutane, pentaerythritol and dipentaerythritol;
lower alkyl glucosides, in particular those with 1 to 8 carbons in the alkyl group, such as methyl and butyl glucoside;
sugar alcohols with 5 to 12 carbon atoms, such as sorbitol or mannitol,
sugars with 5 to 12 carbon atoms, such as glucose or sucrose;
amino sugars, such as glucamine;
dialkoholamines such as diethanolamine or 2-amino-1,3-propanediol.

Suitable preservatives include for example phenoxyethanol, formaldehyde solution, parabens, pentanediol or sorbic acid as well as the silver complexes known as Surfacine® and the further substance classes listed in Annex 6, Parts A and B of the Cosmetic Regulation (Kosmetikverordnung).

Chewing Gums

Preferred oral preparations may also be chewing gums. These products typically contain a water-insoluble and a water-soluble component.

The water-insoluble base, also known as “gum base”, usually comprises natural or synthetic elastomers, resins, fats and oils, plasticizers, fillers, colorants and optionally waxes. The proportion of the base usually amounts to 5 to 95, preferably 10 to 50 and in particular 20 to 35% by weight of the total composition. In a typical embodiment of the invention, the base is composed of 20 to 60% by weight synthetic elastomers, 0 to 30% by weight natural elastomers, 5 to 55% by weight plasticizers, 4 to 35% by weight fillers and, in minor amounts, additives such as colorants, antioxidants and the like, with the proviso that they are water-soluble only in small amounts.

Suitable synthetic elastomers include polyisobutylenes having average molecular weights (according to GPC) of 10,000 to 100,000 and preferably 50,000 to 80,000, isobutylene-isoprene copolymers (“butyl elastomers”), styrene-butadiene copolymers (styrene:butadiene ratio e.g. 1: 3 to 3: 1), polyvinyl acetates having average molecular weights (according to GPC) of 2,000 to 90,000 and preferably 10,000 to 65,000, polyisoprene, polyethylene, vinyl acetate-vinyl laurate copolymers and mixtures thereof. Examples of suitable natural elastomers are rubbers such as smoked or liquid latex or guayule as well as natural rubber materials such as jelutong, lechi caspi, perillo, sorva, massaranduba balata, massaranduba chocolate, nispero, rosindinba, chicle, gutta hang lkang and mixtures thereof. The choice of synthetic and natural elastomers and their mixing ratios depends essentially on whether the chewing gums are to be used for producing bubbles (“bubble gums”) or not. Elastomer mixtures containing jelutong, chicle, sorva and massaranduba are preferably used.

In most cases the elastomers are found to be too hard or too little deformable during processing, so it has turned out to be advantageous to use special plasticizers, which of course also have to meet all requirements for approval as food additives. In this respect, esters of resin acids, such as esters of lower aliphatic alcohols or polyols having wholly or partially cured monomeric or oligomeric resin acids, are particularly suitable. In particular, methyl, glycerol or pentareythritol esters and their mixtures are used for this purpose. Alternatively, terpene resins, which can be derived from alpha-pinene, beta-pinene, delta-limonene or mixtures thereof, can also be used.

Suitable fillers or texturing agents are magnesium or calcium carbonate, ground pumice stone, silicates, especially magnesium or aluminum silicates, clays, aluminum oxides, talc, titanium dioxide, mono-, di- and tricalcium phosphate and cellulose polymers.

Suitable emulsifiers are talc, hardened talc, hardened or partially hardened vegetable oils, cocoa butter, partial glycerides, lecithin, triacetin and saturated or unsaturated fatty acids having 6 to 22 and preferably 12 to 18 carbon atoms as well as their mixtures.

FD and C types approved for coloring food, plant and fruit extracts as well as titanium dioxide can for example be used as colorants and whiteners.

The base materials may contain waxes or be wax-free; examples of wax-free compositions can be found in patent specification US 5,286,500, which content is herewith explicitly referred to.

In addition to the water-insoluble gum base, chewing gum preparations regularly contain a water-soluble part which is formed, for example, by plasticizers, sweeteners, fillers, flavorings, flavor enhancers, emulsifiers, dyes, acidifiers, antioxidants and the like, here with the proviso that the components have at least sufficient water solubility. Depending on the water solubility of the particular components, individual components may belong to both the water-insoluble and water-soluble phases. However, it is also possible to use combinations of, for example, a water-soluble and a water-insoluble emulsifier, whereby the individual components are then in different phases. Usually the water-insoluble part makes up 5 to 95 and preferably 20 to 80% by weight of the preparation.

Water-soluble softeners are added to chewing gum preparations to improve chewability and chewing sensation and are typically present in mixtures in amounts of 0.5 to 15% by weight. Typical examples are glycerol, lecithin and aqueous solutions of sorbitol, cured starch hydrolysates or grain syrup.

Sweet Substance can be either sugar-containing or sugar-free compounds, which are used in amounts of 5 to 95, preferably 20 to 80 and in particular 30 to 60% by weight based on the chewing gum composition. Typical saccharide sweeteners are sucrose, dextrose, maltose, dextrin, dried invert sugar, fructose, levulose, galactose, corn syrup and their mixtures. Sorbitol, mannitol, xylitol, hardened starch hydrolysates, maltitol and mixtures thereof can be used as sugar substitutes. Furthermore, HIAS (“High Intensity Articifical Sweeteners”), such as sucralose, aspartame, acesulfame salts, alitame, saccharin and saccharin salts, cyclamic acid and its salts, glycyrrhizine, dihydrochalcone, thaumatin, monellin and the like, alone or in blends, may also be used as additives. Particularly effective are also hydrophobic HIAS, which are the subject matter of the international patent application WO 2002 091849 A1 (Wrigleys) as well as stevia extracts and their active ingredients, in particular rebaudioside A. The amount of these substances used depends primarily on their effectiveness and is typically in the range of 0.02 to 8% by weight.

Fillers such as polydextrose, rafftilose, rafitilin, fructo oligosaccharides (NutraFlora), palatinose oligosaaccharides, guar gum hydrolysates (Sun Fiber) and dextrins are particularly suitable for the production of low-calorie chewing gums.

The selection of other flavoring substances is practically unlimited and uncritical for the essence of the invention. Usually the total proportion of all flavoring substances is 0.1 to 15 and preferably 0.2 to 5% by weight relative to the chewing gum composition. Other suitable flavoring substances include essential oils, synthetic flavours and the like, such as anise oil, star anise oil, caraway oil, eucalyptus oil, fennel oil, lemon oil, wintergreen oil, clove oil, and the like, which are also used, for example, in oral and dental care products.

The chewing gums may further contain auxiliaries and additives, which are particularly suitable for dental care and to fight plaque and gingivitis, such as chlorhexidine, CPC or triclosan. Furthermore, pH regulators (e.g. buffer or urea), active ingredients against caries (e.g. phosphates or fluorides), biogenic active ingredients (antibodies, enzymes, caffeine, plant extracts) may be contained as long as these substances are approved for food and do not interact with each other in an undesirable manner.

### INDUSTRIAL APPLICABILITY

Another object of the invention concerns a first method for improving the taste quality of phloretin in a foodstuff, characterized in that it comprises the following steps:
(a) providing a food base containing a sweet substance and phloretin; and
(b) adding an amount of naringenin to the base which improves the taste quality of phloretin.

A second method for improving the taste quality of phloretin in a foodstuff is also claimed, characterized in that it comprises the following steps:
(a) providing a food base containing a sweet substance; and
(b) adding an effective amount of phloretin and naringenin to the base, wherein the amount of naringenin is sufficient to improve the taste quality of the phloretin.

An effective amount of naringenin is to be understood to be any amount that is capable of masking or compensating the bitter and astringent taste impression of phloretin. In case of doubt, the skilled person can determine the corresponding amounts by simple testing without further guidance and inventive efforts. As a rule, an effective amount of naringenin corresponds to about one tenth to about ten times the amount of phloretin.

A final object of invention concerns the use of naringenin to improve the taste quality of phloretin, especially when phloretin is present in a foodstuff.

### EXAMPLES

Examples 1-2, Comparison Examples V1-V2

With a panel consisting of 9 experienced testers a blind tasting of different test formulations was carried out. The following properties should be evaluated on a scale from (1)=low to (6) high

instant sweetness (“onset sweetness”)

sweetness intensity (“sweetness itensity”)

fullness of the sugar taste (“Full body, sugar like taste”)

bitterness (“bitter”)

astringency (“astringent”)

The results are summarized in Table 1 below.

TABLE 1
Tasting results

| | | | | Full body | | |
|---|---|---|---|---|---|---|
| | | Onset | Sweetness | sugar like | | |
| Example | Composition | sweetness | intensity | taste | Bitter | Astringent |
| C | Base (5% sugar) | 4.1 | 4.6 | 4.6 | 0.7 | 1.0 |
| V1 | Base + | 4.4 | 4.8 | 4.5 | 1.0 | 1.0 |
| | 15 ppm phloretin | | | | | |
| V2 | Base + | 4.8 | 5.8 | 4.1 | 2.3 | 2.5 |
| | 30 ppm phloretin | | | | | |
| 1 | Base + | 5.2 | 6.0 | 4.6 | 1.6 | 1.7 |
| | 15 ppm phloretin + | | | | | |
| | 15 ppm R/S-naringenin | | | | | |
| 2 | Base + | 5.3 | 6.0 | 4.8 | 1.8 | 2.3 |
| | 30 ppm phloretin + | | | | | |
| | 30 ppm R/S-naringenin | | | | | |

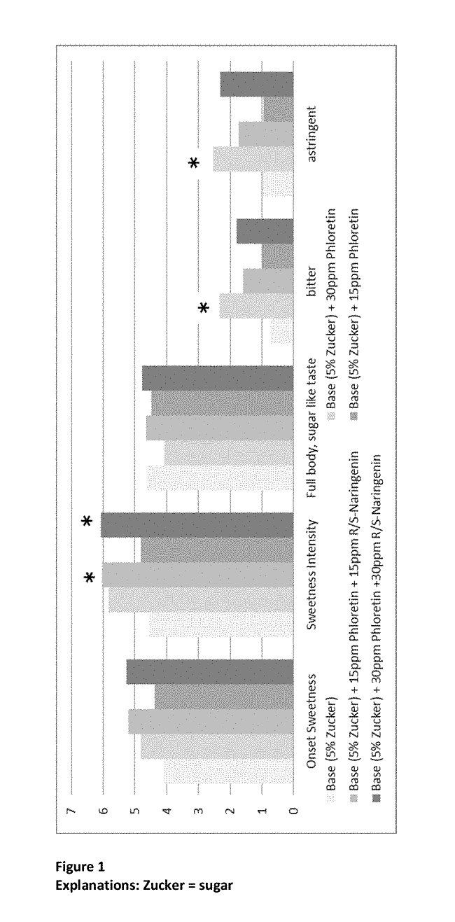

A graphical illustration of the tasting results is shown in FIG. 1 below. The columns marked with (*) show significant differences to the control base (C) (t-test, α=1)

Tables A to G below show exemplary formulations for different end products.

TABLE A
Flavor preparations

| Composition | A | B | C | D | E | F | G | H | I | J |
|---|---|---|---|---|---|---|---|---|---|---|
| R-naringenin | 10 | 20 | 10 | | | | | 10 | | |
| S-naringenin | | | | 20 | 15 | | 7.5 | | | |
| R/S-naringenin | | | | | | 10 | | | 20 | 20 |
| Hesperetin from citrus | 2 | | | | | | | | 2 | |
| (racemic, >80%) | | | | | | | | | | |
| (S)-Hesperetin from honey | | | 1 | | | | 1.5 | | | |
| bush (>95%) | | | | | | | | | | |
| Phloretin | 10 | 10 | 2 | 5 | | 8 | 2 | 7 | 5 | 10 |
| Hesperetin | | 3 | | 3 | 1 | | | 2 | | 3 |
| 1-(2,4-dihydroxy-phenyl)- | | | | | | 2 | | | | |
| 3-(3-hydroxy-4-methoxy- | | | | | | | | | | |
| phenyl)-propan-1-one | | | | | | | | | | |
| 7,3-dihydroxy- | | | | | 2 | | | | | |
| 4′methoxyflayan | | | | | | | | | | |
| 5-Hydroxy-4-(4-hydroxy-3- | | | | | | 2 | | | | |
| methoxy-phenyl)-7- | | | | | | | | | | |
| methoxy-2-chromanone | | | | | | | | | | |
| Glycerol | 10 | 20 | | 30 | | 20 | 15 | 30 | 10 | 20 |
| Gum arabic solution (20%) | | | | | | | | 10 | | |

| | |
|---|---|
| 1,2-Propylene glycol | Ad 100 |

TABLE B
Soft drinks (amounts as % by weight)

| Ingredients | A | B | C | D | E | F | G |
|---|---|---|---|---|---|---|---|
| Sucrose | 10 | 10 | 7 | — | — | 8 | 7 |
| Glucose/fructose Syrup | — | — | — | — | 10 | — | — |
| Phloretin/naringenin | 0.003 | 0.005 | 0.003 | 0.01 | 0.0045 | 0.0025 | 0.001 |
| mixture A from example | | | | | | | |
| 1 | | | | | | | |
| Citric acid | 0.15 | 0.15 | 0.06 | 0.15 | 0.15 | 0.15 | 0.15 |
| Phosphoric acid | — | — | 0.07 | — | — | — | — |
| Sugar coleur | — | — | 0.14 | — | — | — | — |
| Caffeine | — | — | 0.01 | — | — | — | — |
| Citrus flavor | 0.1 | 0.05 | — | 0.1 | 0.1 | 0.1 | 0.1 |
| Lemon flavor | — | 0.05 | — | — | — | — | — |
| Beverage emulsion type | — | — | 0.05 | — | — | — | — |
| “Cola” | | | | | | | |
| Hesperetin | 0.00075 | 0.0012 | 0.00075 | 0.0012 | 0.00075 | 0.00075 | 0.01 |
| Homoeriodictyol-Na | — | — | 0.005 | 0.005 | — | — | — |

| | |
|---|---|
| Water | Ad 100 |

Production: The ingredients are mixed in the given order and filled up to 100% with water. The mixtures are then filled into glass bottles and carbonated.

TABLE C
Hard caramels (amounts as % by weight)

| Ingredients | A | B | C | D |
|---|---|---|---|---|
| Sugar | 74.50 | — | — | — |
| Palatinite, type M | — | 74.00 | 75.50 | 75.00 |
| Citric acid | 0.5 | 1.0 | 0.5 | — |
| Colorant, yellow | — | 0.01 | — | — |
| Colorant, red | — | — | 0.01 | — |
| Colorant, blue | 0.01 | — | — | 0.01 |
| Peppermint flavor | 0.1 | — | — | 0.1 |
| Citrus flavor | — | 0.1 | — | — |
| Red beery flavor | — | — | 0.1 | — |
| Phloretin/naringenin | 0.005 | 0.0045 | 0.006 | 0.003 |
| mixture A from | | | | |
| Example 1 | | | | |
| Balansin A | — | 0.005 | 0.010 | 0.005 |
| Hesperetin | 0.00075 | 0.001 | 0.0005 | 0.0004 |
| Water | ad 100 | ad 100 | ad 100 | ad 100 |

TABLE D
Yoghurt with low fat content (amounts as % by weight)

| Ingredients | A | B | C | D |
|---|---|---|---|---|
| Sucrose | 10 | 8 | 6 | — |
| Sucralose | — | 0.02 | — | 0.2 |
| Rebaudioside A > 95% | — | — | 0.025 | — |
| Saccharin | — | — | — | 0.3 |
| Phloretin/naringenin mixture A from | 0.005 | 0.0045 | 0.01 | 0.050 |
| Example 1 | | | | |
| Sour cherry extract from Example 1 | 0.2 | 0.1 | 0.2 | 0.2 |
| Hesperetin | 0.00075 | 0.001 | 0.003 | 0.002 |
| Homoeriodictyol sodium salt | — | — | — | 0.005 |

| | |
|---|---|
| Yoghurt, 0.1% fat | Fill up to 100% |

TABLE E
Fruit gums (amounts as % by weight)

| Ingredients | A | B |
|---|---|---|
| Sucrose | 34.50 | 8.20 |
| Glucose syrup, DE 40 | 31.89 | 30.09 |
| Phloretin/naringenin mixture A from Example 1 | 0.005 | 0.0045 |
| Iso syrup C* Tru Sweet 01750 (Cerestar GmbH) | 1.50 | 2.10 |
| Gelatine 240 Bloom | 8.20 | 9.40 |
| Polydextrose (Litesse ® Ultra. Danisco Cultor GmbH) | — | 24.40 |
| Colorant | 0.01 | 0.01 |
| Citrus flavor | 0.20 | — |
| Cherry flavor | — | 0.10 |
| Hesperetin | 0.0075 | 0.0015 |
| Water | ad 100 | ad 100 |

TABLE F
Sugar-free chewing gum (amounts as % by weight)

| Ingredients | Content |
|---|---|
| Chewing gum base | 30.00 |
| Sorbitol powder | Ad 100 |
| Phloretin/naringenin mixture A from Example 1 | 0.05 |
| Palatinite | 9.50 |
| Xylitol | 2.00 |
| Mannitol | 3.00 |
| Aspartame | 0.10 |
| Acesulfame K | 0.10 |
| Emulgum/emulsifier | 0.30 |
| Sorbitol 70% in water | 14.00 |
| Glycerol | 1.00 |
| Pepper mint flavor | 1.50 |
| Hesperetin | 0.01 |

TABLE G
Pudding (amounts in g)

| Ingredients | A | B | C |
|---|---|---|---|
| Corn starch | 38 | 38 | 38 |
| Sugar | 38 | 30 | 22.8 |
| Phloretin/naringenin mixture A from | — | 0.05 | 0.08 |
| Example 1 | | | |
| Hesperetin | — | 0.01 | 0.03 |
| Vanilla flavor (Symrise) | 0.2 | 0.2 | 0.2 |
| Quinoline yellow | 0.02 | 0.02 | 0.02 |
| Milk | 500 ml | 500 ml | 500 ml |

1-18. (canceled)

19. Substance mixtures, comprising
(a) phloretin,
(b) naringenin,
(c) at least one additional sweet substance, which is different from phloretin and naringenin, as well as optionally
(d) at least one flavouring, wherein the components (a) and (b) are present in a weight ratio of 99:1 to 1:99.

20. The substance mixtures according to claim 19, characterized in that the sweet substances, which form the component (c), are selected from the group consisting of saccharose, trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyd, maltodextrine of different polymerisation grades as well as plant-based preparations, which contain these substances.

21. The substance mixtures according to claim 19, characterized in that the sweet substances, which form the component (c), are selected from at least one of the following groups:
(i) enzymatic produced starch or sugar hydrolysates such as invert sugar or fructose syrup;
(ii) fruit concentrates;
(iii) sugar alcohols such as erythritol, threitol, arabitol, ribotol, xylitol, sorbitol, mannitol, dulcitol or lactitol;
(iv) sweet tasting proteins and petides such as miraculin, monellin, thaumatin, curculin or brazzein;
(v) synthetic sweeteners such as magap, sodium cyclamat, acesulfam K, neohesperidin hydrochalcone, saccharin sodium salt, aspartame, super-aspartame , neotame, alitame, sucralose, steviosides, rebaudiosides, rubusosides, suavio sides, mogrosides, lugdunames, carrelames, sucronates, sucrooactates, monatines, phenylodulcines, phyllodulcines or abrusosides;
(vi) naturally occurring sweeteners such as stevioside, steviolbioside, rebaudioside A, further steviolglycosides such as rebaudioside B, rebaudioside C, rebaudioside D, rebaudioside E, rebaudioside F, rebadioses G, rebaudioside H, culcoside and/or rubusoside, oslandin, polypodoside A, strogin 1, strogin 2, strogin 4, selligueanin A, dihydroquercetine-3-acetate, perillartin, telosmoside A15, periandrin I-V, phyllodulcin, pterocaryosides, cyclocaryosides, mukurisiosides, trans-anethole, trans-cinnamaldehyde, bryosides, bryonosides, bryonodulcosides, carnosiflosides, scandenosides, gypenosides, trilobatin, phloridzine, dihydroflavanoles, hematoxylin, cyanin, chlorogenic acid, albiziasaponin, telosmosides, gaudichaudio side, mogrosides, hernandulcine, glycyrrhetinic acid, balansin A, balansin B;
(vii) extracts or enriched fractions of these extracts such as thaumatococcus extracts (Katemfestaude), extracts from Stevia ss. (especially Stevia rebaudiana), swingle extract (Momordica or Siratia grosvenorii, Luo-Han-Guo), extracts from liquorice root, also Glycerrhyzia ssp. (especially Glycerrhyzia glabra), Rubus ssp. (especially Rubus suavissimus), citrus extracts, extracts from Hydrangea macrophyll var. thunbergii Makino, extacts from Lippia dulcis, extracts from Mycetia balansae;
(viii) sweet tasting amino acids (e.g. glycine, D-leucine, D-threonine, D-asparagine, D-phnylalanine, D-Tryptophane, L-proline);
(ix) sweet tasting low molecular weight substances, such as hernandulcin, dihydro chalconglykosides, glycyrrhizine, glycerrhetinic acid, derivates and salts thereof, extracts from liquorice (Glycyrrhizza glabra ssp.), Lippia dulcis extracts, Momordica ssp. extracts or fruit preparations or extracts e.g. from Momordica grosvenori [Luo Han Guo], Hydrangea dulcis, Stevia ssp. (e.g. Stevia rebaudiana);
(x) enzymatically treated glycosidic sweeteners or extracts from Stevia ssp. or Rubus ssp; as well as
(xi) extracts or isolated balansines from Mycetia balansae.

22. The substance mixture according to claim 19, characterized in that they contain flavourings as optional compound (d), which are selected from the group consisting of acetophenone, allylcapronate, alpha-ionon, beta-ionon, anisaldehyde, anisylacetate, anislformate, benzaldehyde, benzothazol, bezylactetate, benzylalcohol, benzylbenzoate, butylbutyrate, butylcapronate, butylidenphtalid, carvon, camphene, caryophyllen, cineol, cinnamylacetate, citral, citronellol, citronellal, citronellylacetat, cyclohexylacetat, cymol, damascon, delta-decalactone, diacetyl, dihydrocumarin, dimethyl anthranilate, dodecalactone, acetic acid, ethylacetate, ethyl butyric acid, ethylbutyrate, ethyl caprinate, ethyl capronate, ethylcrotonate, ethylfuraneol, ethylguajakol, ethylisobutyrat, ethylisovalerianate, ethyllactate, ethylmethylbutyrate, ethylpropionate, eucaloptol, eugenol, ethylheptylate, geraniol, geranylacetate, methyldihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenic acid, trans-3-hexenic acid, cis-3-hexenylacetate, cis-3-hexenylcapronate, trans-2-hexenylcapronate, cis-3-hexenyo acetate, cis-3-hexenylcapronate, cis-3-hexenylformiate, para-hydroxybenyzyl acetone, isoamylalcohol, isoamylisovalerianate, isobutylbutyrate, isobutyraldehyde, isoeugenolmethylether, isopropylmethylthiazol, lauric acid, levulinic acid, linalool, linalooloxide, linalylacetate, menthol, menthofuran, menthylanthranilate, menthylbutanol, methyl butyric acid, 2-menthylbutylacetate, menthylcapronate, menthylcinnamate, 5-methylfurfural, 3,2,2-menthylbutylacetate, menthylcapronate, mentylcinnamate, 5-methylfurfural, 3,2,2-methylcyclopentenolon, 6,5,2-mentylheptenon, methyljasmonate, 2-mentylmethylbutyrate, 2-menthyl-2-pentenolic acid, methylthiobutyrate, 3,1-methylthiohexanol, 3-methylthiohexylacetate, nerol, nerylacetate, trans,trans-2,4-nonadienal, 2,4-nonadienol, 2,6-nonadienol, nootkaton, delta octalactone, gamma octalactone, 2-octanol, 3-octanol, 1,3-octenol, 1-octylacetate, 3-octylacetate, palmitic acid, paraldehyde, phellandren, pentandion, phenylethylacetate, phenylethylalcohol, phenylethylisovalerianate, propionaldehyde, propylbutyrate, pulegon, pulegol, sinensal, sulfurol, terpinen, terpineol, terpinolen, 8,3-thiomethanone, 4,4,2-thiomethylpentanone, thymol, delta-undecalactone, gamma-undecalactone, valcen, valerianic acid, vanillin, acetoin, ethylvanillin, ethylvannilisobutyrat (=3-ethoxy-4-isobutyryloxybenzaldehyde), 2,5-dimethyl-4-hydroxy-3(2H)-furanone and derivatives thereof (preferably homofuraneol(=2-ethyl-4-hydroxy-5-methyl-3(2H)-furanone), homofuronole (=2-ethyl-5-methyl-4-hydroxy-3(2H)-furanone and 5-ehyl-2-methyl-4-hydroxy-3(2H)-furanone), maltol and maltol-derivates (preferably ethylmaltol), cumarin and cumarin derivatives, gamma-lactones (preferably gamma-undecalactone, gamma-nonalactone, gamma-decalactone), delta-lactones (preferably 4-methyldeltadecalactone, massoilactone, deltadecalactone, tuberolactone), methylsorbate, divanillin, 4-hydroxy-2(or 5)-ethyl-5(or 2)-methyl-3(2H)-furanone, acetic acid amylester, butyric acid isomaylester, 3-methyl-butyric acid ethylester, ethyl-3-methyl-3-phenylglycidate, ethyl-2-trans-4-cis-decadienoate, 1,1-dimethoxy-2,2,5-trimethyl-4-hexane, phenylacetaldehyde, 2-methyl-3-(methylthio)furan, 2-methyl-3-furanthiol, bis(2-methyl-3-furyl)disulphide, furfurylmercaptane, methional, 2-Acetyl-2-thiazoline, bis (2-methyl-3-furyl)disulphide, furfurylmercaptane, methionale, 2-acetyl-2-thiazoline, 3-mercapto-2-pentanone, 2,5-dimethyl-3-furanthil, 2,4,5-trimethylthiazol, 2-acetylthiazol, 2,4-dimethyl-5-ethylthiazol, 2-acetyl-1-pyrollin, 2-methyl-3-ethylpyrazin, 3-isopropyl-2-methoxypyrazin, 3-isobutyl-2-menthoxypyrazin, 2-acetylpyrazin, 2-pentylpyridin, (E,E)-2,4-decadienale, (E,E)-2,4-nonadienal, (E)-2-octenal, (E)-2-nonenal, 2-undecenal, 12-mentyltridecanal, 1-penten-3-on, guajakol, 3-hydroxy-4,5-dimethyl-2(5H)-furanone, 3-hydroxy-4-methyl-5-ethyl-2(5H)-furanone, cinammic aldehyde, cinnamic alcohol, methylsalicyclat, isopulegol as wells as (not explicitly mentioned) stereoisomers, enantiomers, positional isomers, diastereomeres, cis/trans-isomers or epimeres of these substances.

23. The substance mixtures according to claim 19, characterized in that they contain flavourings as optional compound (d), which are selected from the group consisting of alpha-ionon, beta-ionon, benzaldehyde, citral, damascone, delta-decalactone, diacetyl, dimethylanthranilate, dodecalactone, acetic acid, ethyl acetate, ethyl butyrate, ethyl furaneol, ethyl isobutyrate, ethyl isovalerianate, ethyl methylbutyrate, ethylpropionate, geraniol, geranylacetate, heliotropin, trans-2-hexenal, cis-3-hexenol. Cis-3-hexenylacetate, para-hydroxybenzylacetone, isobutyraldehyde, linalool, Linalylacetate, 3,2,2-methylcyclopentenolon, 2-methylmethylbutyrate, nerol, nerylacetate, vanillin, ethylvanillin, 2,5-dimethyl-4-hydroxy-3(2H)-furanone, homofuronol (=2-ethyl-5-methyl-4-hydroxy-3(2H)-furanone and 5-ethyl-2-methyl-4-hydroxy-3(2H)-furanone), maltol and maltol derivatives (preferably ethylmaltol), gamma-lactone (preferably 4-methyldeltadecalactone, massoilactone, deltadecalactone, tuberolactone), 4-hydroxy-2(or 5)-ethyl-5(or 2)-methyl-3(2H)furanone, 2-hydroxy-3-methyl-2-cyclopentenone,3-hydroxy-4,5-dimethyl-2(5H)-furanone, acetic acid isoamylester, butyric acid isoamylester, 3-methyl-butyric acid ethylester, ethyl-2-trans-4-cis-decadienoate, (E,E)-2,4-decadienal, guajakol, cinnamic aldehyde as well as (not explicitly mentioned) stereoisomers, enantiomers, positional isomers, diastereomeres, cis/trans-isomers or epimers of these substances.

24. The substance mixtures according to claim 19, characterized in that they contain flavourings as optional compound (d), which are selected from the group consisting of adenosine-5′-monophosphate, cytodine-5′-monophosphate, inosin-5′-monophosphate, and their pharmaceutically acceptable salts; lactisolen; 2,4-dihydroxynezoic acid; 3-hydroxybenzoic acid; sodium salts preferably sodium chloride, sodium lactate, sodium citrate sodium acetate, sodium gluconoate; hydroxyflavanones, such as for example eriodictyol, homoeriodictyol, and their sodium salts; hydroxybenzoic acid amides, such as for example 2,4-dihydroxybenzoic acid vanillylamide, 2,4-Di-hydroxybenzoic acid-N-(4-hydroxy-3-methoxybezyl)amide, 2,4,6-trihydroxy-benzoic acid-N-(4-hydroxy-3-methoxybenzyl)amide, 2-hydroxy-benzoic acid-N-4-(hydroxyl-3-methoxybenzyl)amide, 4-hydroxybenzoic acid-N-(4-hydroxy-3-methoxybenzyl)-amide, 2,4-dihydroxy-benzoic acid-N-(4-hydroxy-3-methoxybenzyl)amide-mono-sodium salt, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide and 2-hydroxy-5-methoxy-N-[2-(4-hydroxy-3-methoxyphenyl)ethyl]amide; 4-hydroxy-benzoic acid vanillylamide; hydroxydeoxybenzoinen, such as for example 2-(4-hydroxy-3-methoxyphenyl)-1-(2,4,6-trihydroxyphenyl)ethanone, 1-(2,4-dihydroxypheyl)-2-(4-hydroxy-3-methoxyphenyl)ethanone, 1-(2-hydroxy-4-methoxyphenyl)-2-(4-hydroxy-3-methoxyphenyl)ethanon); hydroxyphenylalkanones, such as for example gingerdione-[2], gingerdione-[3], gingerdione-[4], dehydrogingerdione-[2], dehydrogingerdione-[3], dehydrogingerdione-[4]); diacetyltrimers; y-amino butyric acid and divanillins;
bicyclo[4.1.0]heptan-7-carbonic acid amides; cyclopropancarbonic acid (3-methyl-cyclohexyl)amides, aromatic Neo-menthyl-amides; geranylamine derivatives of oxalic acid as well as neomenthyl derivatives and mixtures thereof.

25. The substance mixtures according to claim 19, characterized in that they contain
(a) 1 to 25% by weight phloretin,
(b) 1 to 25% by weight naringenin, and
(c) 1 to 50% by weight of at least one further sweet substance, which is different from phloretin and naringenin,
with the proviso that the indications of quantity, optionally with the addition of flavourings, which form the optional component (d), summarize to 100% by weight.

26. The substance mixtures according to claim 19, characterized in that they contain with regard to the components (a) and (b) alone
(a) 5 to 99.8% by weight phloretin,
(b1) 0.1 to 94.9% by weight S-naringenin, and
(b2) 0.1 to 94.9% by weight R-naringenin, wherein
in the proviso that one of the naringenin-enantiomers can be present with regard to the total content of naringenin in a weight ratio from 100:1 to 1:100 and that the indication of quantities summarizes to 100% by weight.

27. The substance mixtures according to claim 19, characterized in that they contain with regard to the componets (a) and (b) alone
(a) 5 to 90% by weight phloretin,
(b1) 10 to 85% by weight S-naringenin, and
(b2) 0.1 to 85% by weight R-narringenin, wherein, with the proviso that the indications of quantities summarize to 100 weight-%.

28. The substance mixtures according to claim 19, characterized in that they contain the components (a+b) and (c) in a weight ratio of 20:80 to 80:20.

29. Oral preparations, comprising the substance mixtures according to claim 19.

30. The preparations according to claim 29, characterized in that they contain the substance mixtures in quantities of 0.00001 to 2% by weight.

31. Use of naringenin to improve the taste quality of phloretin.
